(19) United States
(12) Patent Application Publication
(10) Pub. No.: US 2005/0187517 A1
(43) Pub. Date: Aug. 25, 2005

(54) Inflation adaptor with alignment grooves

(75) Inventors: Gregory R. Dion, Tucson, AZ (US); Thierry H. Benjamin, Lowell, MA (US)

Correspondence Address: JAMES F. CRITTENDEN;MEDTRONIC VASCULAR, INC., 37A CHERRY HILL DRIVE, DANVERS, MA, 01923, US

(73) Assignee: MEDTRONIC VASCULAR, INC.

(21) Appl. No.: 10/784,441

(22) Filed: Feb. 23, 2004

(51) Int. Cl. A61M 29/00

(52) U.S. Cl. 604/99.01; 606/192

(57) ABSTRACT

A system for treating a vascular condition, including an inflation adaptor having a housing, a clamping device positioned within the housing, and a medial v-groove alignment block. The clamping device includes a jaw and an anvil. An extended portion of a valve stem and a hollow guidewire are received in the medial v-groove alignment block. The extended valve stem and hollow guidewire are engaged by the clamping device to allow the extended valve stem and the hollow guidewire to be axially translated relative to each other to control a flow of an inflation fluid through the hollow guidewire when the clamping device is in a clamped position. A method of operation is also disclosed.

### FIELD OF THE INVENTION

This invention relates generally to balloon catheters and guidewire deployment of catheter-based treatment tools. More specifically, the invention relates to an inflation adaptor with alignment blocks for an occlusion catheter.

### BACKGROUND OF THE INVENTION

One or more treatment catheters may be introduced over and removed from a single guidewire during intravascular treatment procedures such as coronary angioplasty or stent placement. These catheter systems have been developed to facilitate easy and quick exchange of wires and catheters during these medical procedures.

Guidewires are used conventionally to guide the insertion of various medical instruments such as catheters to a desired treatment location within a patient's vasculature. In a typical procedure, the clinician forms an access point for the guidewire by creating an opening in a peripheral blood vessel, such as the femoral artery. The flexible guidewire is introduced through the opening into the peripheral blood vessel, and advanced by the clinician through the patient's blood vessels until the guidewire extends across the vessel segment to be treated. Various treatment catheters, such as a balloon dilatation catheter for a percutaneous transluminal coronary angioplasty, are inserted over the guidewire and similarly advanced through vasculature until they reach the treatment site.

The guidewire may be hollow with an inflatable member such as a balloon mounted at its distal end, and an inflation lumen between the inflatable member and an inflation port at its proximal end.

Hollow guidewires have been designed with low-profile valves that can be opened and closed by detachable inflation adaptors to control the passage of inflation fluid to and from balloons mounted on the guidewires. Such a balloon guidewire is typically connected to a removable inflation manifold or inflation adaptor and includes an integral valve to maintain the balloon in its inflated state when the manifold is removed. A low-profile catheter valve is advantageous for use with occlusion guidewires, as well as therapeutic or anchorable devices that may have outer diameters of 0.014 inches or smaller. Further details regarding catheter valves, catheter balloons, and inflation adaptors are found in “Low Profile Catheter Valve and Inflation Adaptor,” Zadno-Azizi et al., U.S. Patent Publication No. 2002/0133117 published Sep. 19, 2002; “Low Profile Catheter Valve and Inflation Adaptor,” Zadno-Azizi et al., U.S. Pat. No. 6,325,778; and “Guidewire Inflation System,” Zadno-Azizi et al., U.S. Pat. No. 6,050,972, all of which are hereby incorporated by reference in their entirety. Details of various inflation adaptors are found in “Inflation Adaptor and Method of Use,” pending U.S. patent publication 10/348,046 filed Jan. 17, 2003, the contents of which are hereby incorporated by reference in their entirety. An integrated inflation/deflation device for delivery of inflation fluid is described in “Integrated Inflation/Deflation Device and Method,” Bagaoisan, et al., U.S. Pat. No. 6,234,996, the contents of which are hereby incorporated by reference in their entirety.

An exemplary inflation adaptor, which may be attached to a low-profile catheter or a hollow guidewire, provides a fluid-tight chamber for introduction of a pressurized fluid that expands a catheter balloon. The inflation chamber releaseably seals its inflation inlet to the inflation port of an elongate, hollow guidewire to form a fluid passage there between. Fluid is supplied to the inflation port under pressure via the fluid passageway. The inflation adaptor also releaseably grips or clamps portions of the hollow guidewire for sliding operation of a valve that controls the flow of inflation fluid to inflate and deflate a catheter balloon. The adaptor may be detached from the hollow guidewire without deflating the balloon, and the balloon remains inflated until the adaptor is again attached to the catheter, the valve is opened, and the inflation fluid is removed.

An inflation adapter may include a channel for transversely receiving the guidewire, and clips or guides to help align the guidewire within the channel prior to clamping and sealing the inflation adapter about the hollow guidewire. When a hollow guidewire is transversely inserted in an exemplary adaptor, an inflation port in the guidewire lies within the fluid-tight inflation chamber of the adaptor. The alignment of the flexible, hollow guidewires and small valve stems in the adaptor is critical for a balloon catheter system to work properly. During transverse loading of the flexible hollow guidewire into the inflation adapter, the guidewire may become bent, kinked and/or misaligned with the clamps and seals prior to closing the adapter about the guidewire.

What is desirable is an improved inflation adaptor that provides faster and easier transverse insertion and alignment of a valve stem and a hollow guidewire into the inflation adaptor. The inflation adaptor should allow for easy transverse loading of a valve stem and a hollow guidewire into the adaptor while avoiding bending or malformation of the valve stem or the hollow guidewire shaft. The inflation adaptor should also provide simple, repeatable positioning of the hollow guidewire within the adapter prior to closing the adapter about the guidewire. A hollow guidewire that is undamaged and correctly positioned within an inflation adapter will provide predictable control of fluid through the hollow guidewire and increased utility and performance of associated medical devices used during the treatment of vascular conditions.

### SUMMARY OF THE INVENTION

One aspect of the invention provides a system for treating a vascular condition including an inflation adaptor having a housing, a clamping device positioned within the housing, and a medial v-groove alignment block. The clamping device includes a jaw and an anvil. A portion of an extended valve stem and a hollow guidewire are received in the medial v-groove alignment block. The extended valve stem and hollow guidewire are engaged by the clamping device to allow the extended valve stem and the hollow guidewire to be axially translated relative to each other to control a flow of an inflation fluid through the hollow guidewire when the clamping device is in a clamped position.

Another aspect of the invention is a method of operating an inflation adaptor. The inflation adaptor is positioned to receive a valve stem partially extended from a proximal end of a hollow guidewire to define a first valve configuration. The extended valve stem and the hollow guidewire are inserted into a medial v-groove alignment block. A portion of the extended valve stem and the hollow guidewire are clamped within the inflation adaptor. The valve stem and the hollow guidewire are relatively translated to a second valve configuration to control the flow of an inflation fluid through a portion of the hollow guidewire.

The present invention is illustrated by the accompanying drawings of various embodiments and the detailed description given below. The drawings should not be taken to limit the invention to the specific embodiments, but are for explanation and understanding. The detailed description and drawings are merely illustrative of the invention rather than limiting, the scope of the invention being defined by the appended claims and equivalents thereof. The foregoing aspects and other attendant advantages of the present invention will become more readily appreciated by the detailed description taken in conjunction with the accompanying drawings, which are not to scale.

### BRIEF DESCRIPTION OF THE DRAWINGS

Various embodiments of the present invention are illustrated by the accompanying figures, wherein:

FIG. 1 is an illustrative view of a system for treating a vascular condition, in accordance with one embodiment of the current invention;

FIG. 2a is a longitudinal cross-sectional view of a portion of a plug valve in a closed position, in accordance with one embodiment of the current invention;

FIG. 2b is a longitudinal cross-sectional view of a portion of a plug valve in an open position, in accordance with one embodiment of the current invention;

FIG. 3 is a perspective view of a medial v-groove alignment block, in accordance with one embodiment of the current invention;

FIG. 4 is a perspective view of a distal v-groove alignment block, in accordance with one embodiment of the current invention;

FIG. 5 is a perspective view of a proximal v-groove alignment block, in accordance with one embodiment of the current invention;

FIG. 6 is an illustrative perspective view of an inflation adaptor including a medial v-groove alignment block, a distal v-groove alignment block and a proximal v-groove alignment block, in accordance with one embodiment of the current invention;

FIG. 7 is a perspective view of a jaw for an inflation adaptor, in accordance with one embodiment of the current invention;

FIG. 8 is a perspective view of an anvil for an inflation adaptor, in accordance with one embodiment of the current invention;

FIG. 9 is an illustrative perspective view of an inflation adaptor including a distal v-groove alignment block, a medial v-groove alignment block, a proximal v-groove alignment block, a jaw and an anvil, in accordance with one embodiment of the current invention;

FIG. 10 is a transverse cross-sectional view of a medial v-groove alignment block with a tapered section of a jaw that mates with the medial v-groove alignment block, in accordance with one embodiment of the current invention; and

FIG. 11 is a flow diagram of a method for operating an inflation adaptor, in accordance with one embodiment of the current invention.

### DETAILED DESCRIPTION OF THE PRESENTLY PREFERRED EMBODIMENTS

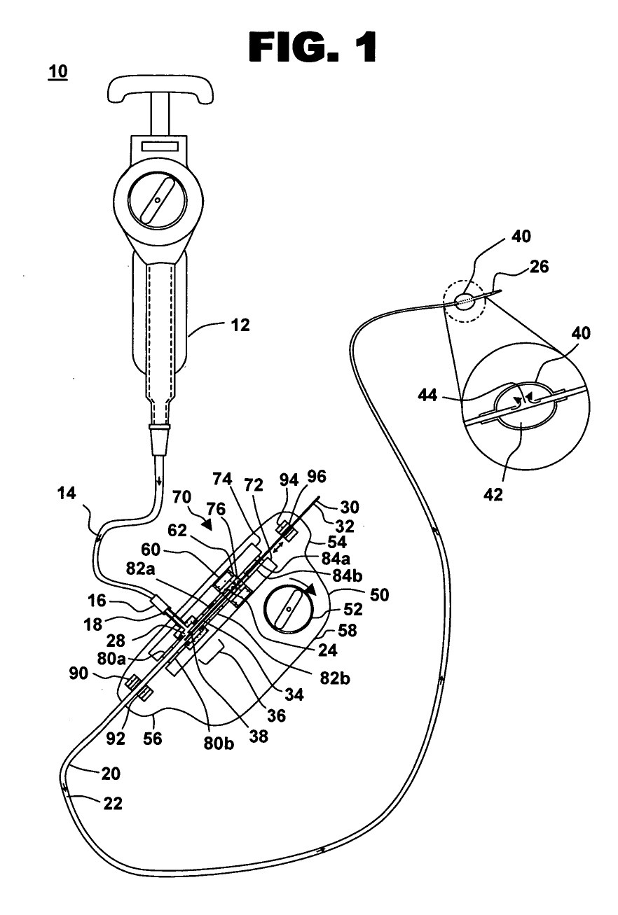

FIG. 1 illustrates a system for treating a vascular condition, in accordance with one embodiment of the present invention. Vascular condition treatment system 10 includes hollow guidewire 20 having central lumen 22, valve stem 30, inflatable balloon 40, and detachable inflation adaptor 50, the latter having a medial v-groove alignment block 60 and clamping device 70. Clamping device 70 including jaw 72 and anvil 74 is positioned within housing 58 of inflation adaptor 50. Inflatable balloon 40, which is attached proximate to distal end 26 of hollow guidewire 20, may be inflated with Inflation fluid 14 from inflation fluid supply 12 that is delivered via central lumen 22 to inflatable balloon 40, the delivery controlled in part by inflation adaptor 50.

For various medical procedures, inflatable balloon 40 comprises, for example, an occlusion balloon, an angioplasty balloon, or a stent-deployment balloon. Vascular condition treatment system 10 may be used, for example, as a temporary occlusion device for blocking fluid flow through arteries or veins. In another medical procedure, vascular condition treatment system 10 is used as a dilatation catheter whereby blood vessels with stenoses may be enlarged by inflating inflatable balloon 40 within the blockage. In other applications, vascular condition treatment system 10 is used in coordination with other treatment catheters, such as a stent-delivery catheter, an aspiration catheter, an inspection catheter, a measurement catheter, an angioplasty catheter, an atherectomy catheter, a drug-delivery catheter, an ultrasound device, a measurement device, a laser catheter, an imaging catheter, a treatment catheter or a therapy catheter. The treatment of vascular conditions may include the prevention or correction of various ailments and deficiencies associated with the cardiovascular system, the cerebrovascular system, and other blood vessels within the body.

Inflation fluid supply 12 with inflation fluid 14 may be coupled to inflation adaptor 50 with suitable fitting 16, which connects to inflation fluid supply port 18 of inflation adaptor 50. Inflation fluid supply port 18 may be a removable fluid fitting such as a Luer fitting. Inflation fluid 14 may be a saline solution, a radiopaque contrast fluid, a dilute contrast agent, or other suitable liquid that is injectable into inflatable balloon 40.

Hollow guidewire 20 is an elongate, flexible tubular member that is inserted into the body to aid in the treatment of various vascular conditions and obstructions in blood vessels such as atherosclerosis, thrombosis and restenosis. Hollow guidewire 20 may be formed, for example, from an extruded or welded tubular material such as nitinol, stainless steel, or other suitable tubing material. In one example, hollow guidewire 20 has an outer diameter of 0.014 inches and an inner diameter on the order of 0.009 inches, with a length between 135 centimeters and 300 centimeters. The length of hollow guidewire 20 may be on the order of 300 centimeters, allowing over-the-wire (OTW) catheters to be inserted into the body once hollow guidewire 20 is in place. In another example, hollow guidewire 20 may be on the order of 175 centimeters in length, suitable for guiding treatment catheters of the rapid-exchange, telescope, multi-exchange and/or zipper types, as are known to those of skill in the art.

Central lumen 22 within hollow guidewire 20 transports inflation fluid 14 between inflation adaptor 50 and inflatable balloon 40. Hollow guidewire 20 has proximal end 24 and distal end 26. Valve port 28, which is located on a sidewall of hollow guidewire 20 near proximal end 24, allows inflation fluid 14 to flow to and from attached inflation adaptor 50, through central lumen 22 of hollow guidewire 20, and to and from inflatable balloon 40. Valve port 28 is positioned to fluidly communicate with inflation fluid supply port 18 while a fluid-tight seal contacts the entire circumference of portions of hollow guidewire 20 that are distal to and proximal to valve port 28. Inflation fluid 14 is injected into interior region 42 of inflatable balloon 40 through balloon inflation hole 44 in a sidewall of hollow guidewire 20. Balloon inflation hole 44 may include, for example, one or more holes or apertures formed in the sidewall of hollow guidewire 20, one or more slits in the sidewall, or a spiraling slot cut into the sidewall. At distal end 26 of hollow guidewire 20, central lumen 22 of hollow guidewire 20 may be plugged or capped to prevent fluid from exiting distal end 26. Additional structures may be added to distal end 26 of hollow guidewire 20, such as a metallic coil or other flexible tubular element, to assist in guiding hollow guidewire 20 through the body. Radiopaque markers and other indicia for determining the location of inflatable balloon 40 also may be added onto hollow guidewire 20.

Valve stem 30 comprises a small diameter, flexible wire slidably received within central lumen 22 of hollow guidewire 20. Extended portion 32 of valve stem 30 extends outwardly from proximal end 24 of hollow guidewire 20. Axially received portion 34 of valve stem 30 is positioned within central lumen 22 of hollow guidewire 20 near proximal end 24. Valve stem 30 comprises, for example, a small-diameter wire of stainless steel, nitinol, or other suitably flexible and strong material. Valve stem 30 may comprise a polymeric material such as nylon or Teflon®, which has good flexibility and sealing properties yet has sufficient rigidity to controllably translate valve stem 30 and valve plug 38 within hollow guidewire 20. A portion of valve stem 30 received within hollow guidewire 20, for example, is sinusoidally shaped to provide a desired degree of friction between valve stem 30 and an interior surface of hollow guidewire 20.

Exemplary plug valve 36 includes valve plug 38 attached to axially received portion 34 of valve stem 30. Plug valve 36 may be in an open position or a closed position when valve stem 30 and hollow guidewire 20 are translated relative to each other. The position of valve plug 38 with respect to valve port 28 controls the flow of inflation fluid 14 into and out of inflatable balloon 40. Valve plug 38 has an interference fit with an interior surface of hollow guidewire 20 to provide a fluid seal. Valve plug 38 is formed from a polymeric material such as polyurethane, an epoxy, a silicone, or a semi-compliant polymer with good sealing and wear-resistant properties. In a first valve configuration, valve plug 38 is positioned distal to valve port 28 of hollow guidewire 20, and plug valve 36 is in a closed position such that fluid flow is blocked. In a second valve configuration, valve plug 38 is positioned proximal to valve port 28 of hollow guidewire 20, plug valve 36 is in an open position and fluid flow is allowed to flow. Extended portion 32 of valve stem 30 and valve plug 38 are translated relative to hollow guidewire 20 to control a flow of inflation fluid 14 into inflatable balloon 40 by blocking or allowing flow through central lumen 22 of hollow guidewire 20. Pushing valve stem 30 into hollow guidewire 20 past valve port 28 prevents inflation fluid 14 from flowing into or out of inflatable balloon 40, allowing inflatable balloon 40 to remain inflated when inflation adaptor 50 is removed and other treatment catheters are positioned over hollow guidewire 20 and guided to a treatment location. Re-attaching inflation adaptor 50 and pulling valve stem 30 so that valve plug 38 is positioned proximal to valve port 28 allow fluid in interior region 42 to be removed from inflatable balloon 40. When inflatable balloon 40 is deflated, valve plug 38 is placed in the closed position to retain some fluid within central lumen 22, and to avoid air and other gases from entering hollow guidewire 20.

To attach inflation adaptor 50, extended portion 32 of valve stem 30 and a portion of hollow guidewire 20 near proximal end 24 are positioned into v-groove 62 of medial v-groove alignment block 60. Medial v-groove alignment block 60 is located within housing 58 of inflation adaptor 50 to receive extended portion 32 of valve stem 30 and a portion of hollow guidewire 20. Extended portion 32 of valve stem 30 may extend past proximal end 54 of inflation adaptor 50 when its placement is complete. As extended portion 32 of valve stem 30 is transversely inserted into inflation adaptor 50, v-groove 62 of medial v-groove alignment block 60 guides transverse insertion of extended portion 32 of valve stem 30 into medial v-groove alignment block 60. Tapered section 76 of jaw 72 mates with medial v-groove alignment block 60 and slidably directs extended portion 32 of valve stem 30 into medial v-groove alignment block 60.

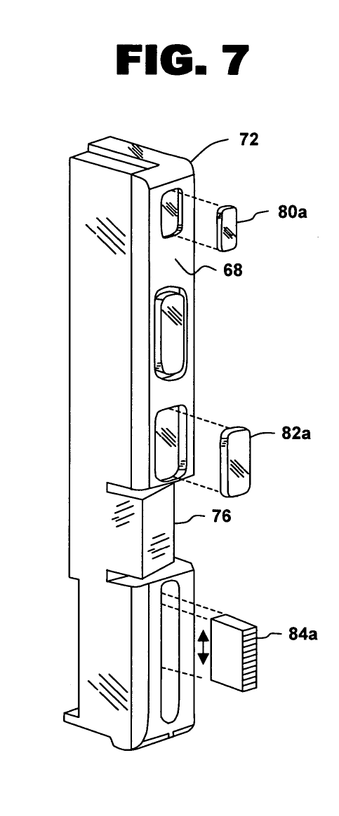

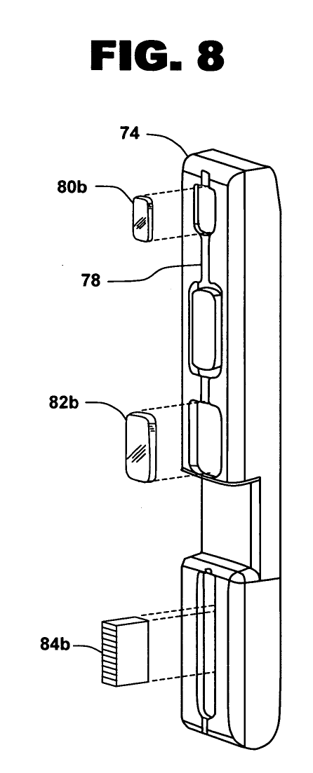

When extended portion 32 of valve stem 30 is transversely inserted into medial v-groove alignment block 60, clamping device 70 may be used for engaging valve stem 30 and hollow guidewire 20. Clamping device 70 is axially aligned with medial v-groove alignment block 60. Clamping device 70 includes a set of distal pads 80a and 80b with frictional surfaces that engage hollow guidewire 20 distal to valve port 28, and a set of medial pads 82a and 82b with frictional surfaces that engage hollow guidewire 20 proximal to valve port 28 when clamping device 70 is in a clamped position. Distal pads 80a, 80b, and medial pads 82a and 82b may have teeth, protrusions, texture, or other features to enhance the gripping of hollow guidewire 20. Clamping device 70 allows valve stem 30 and hollow guidewire 20 to axially move or translate relative to each other when clamping device 70 is in a clamped position, thereby controlling the flow of inflation fluid 14 into inflatable balloon 40. Clamping device 70 includes a set of sliding pads 84a and 84b, as shown in FIGS. 7 and 8, with frictional surfaces that engage extended portion 32 of valve stem 30 proximal to proximal end 24 of hollow guidewire 20 and that control axial translation of valve stem 30 relative to hollow guidewire 20. Sliding pads 84a and 84b may have teeth, protrusions, texture, or other features to enhance the gripping of extended portion 32 of valve stem 30.

The rotation of multi-position actuation knob 52, which is coupled to clamping device 70, allows the axial insertion, engagement, and actuation of plug valve 36, thereby controlling the flow of inflation fluid 14 into inflatable balloon 40. In a first position, actuation knob 52 allows extended portion 32 of valve stem 30 and proximal end 24 of hollow guidewire 20 to be transversely inserted into clamping device 70. In a second position, actuation knob 52 activates clamping device 70 to engage extended portion 32 of valve stem 30 and hollow guidewire 20. In a third position, actuation knob 52 translates valve stem 30 relative to hollow guidewire 20 to control the flow of inflation fluid 14 into and out from inflatable balloon 40.

Inflation fluid 14 from inflation fluid supply 12 that is coupled to inflation adaptor 50 is injected through a portion of hollow guidewire 20 into interior region 42 of inflatable balloon 40 when clamping device 70 is in a clamped position and when plug valve 36 within hollow guidewire 20 is in an open position. Similarly, inflation fluid 14 may be removed from interior region 42 of inflatable balloon 40 when clamping device 70 is in a clamped position and plug valve 36 is in an open position to deflate inflatable balloon 40.

Distal v-groove alignment block 90 including v-groove 92 may be located near distal end 56 of inflation adaptor 50 to guide transverse insertion of hollow guidewire 20 into distal v-groove alignment block 90 as hollow guidewire 20 and extended portion 32 of valve stem 30 are transversely inserted into medial v-groove alignment block 60. Tapered flanks of v-groove 92 slidably direct hollow guidewire 20 into distal v-groove alignment block 90 when hollow guidewire 20 with extended valve stem 30 is transversely inserted into inflation adaptor 50.

A proximal v-groove alignment block 94 including v-groove 96 may be located near proximal end 54 of inflation adaptor 50 to guide transverse insertion of extended portion 32 of valve stem 30 into proximal v-groove alignment block 94 as extended portion 32 of valve stem 30 and hollow guidewire 20 are transversely inserted into medial v-groove alignment block 60. Tapered flanks of v-groove 96 slidably direct extended portion 32 of valve stem 30 into proximal v-groove alignment block 94 when hollow guidewire 20 with extended valve stem 30 is transversely inserted into inflation adaptor 50.

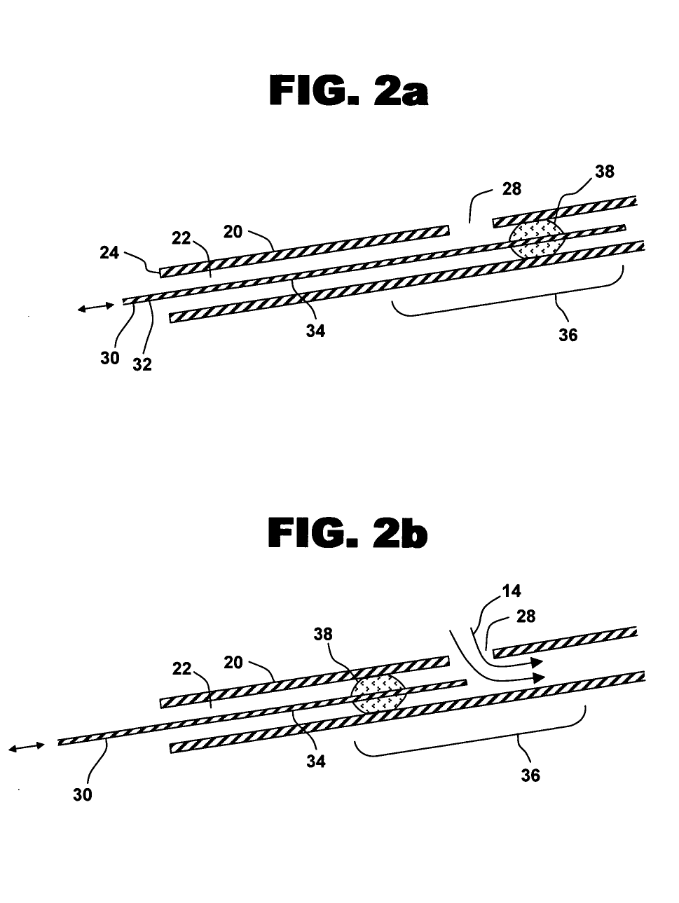

FIG. 2a shows a longitudinal cross-sectional view of a portion of plug valve 36 in a closed position, in accordance with one embodiment of the present invention. In this figure and following figures, like-numbered elements refer to similar elements as in FIG. 1. Plug valve 36 includes extended portion 32 of valve stem 30 extending outwardly from proximal end 24 of hollow guidewire 20. Axially received portion 34 of valve stem 30 extends into a portion of central lumen 22 near proximal end 24 of hollow guidewire 20. Plug valve 36 is positioned in one of an open position or a closed position to control the flow of inflation fluid in central lumen 22 of hollow guidewire 20. Axially translating valve stem 30 of plug valve 36 controls flow of inflation fluid through central lumen 22 of hollow guidewire 20. Valve plug 38 is attached near a distal end of valve stem 30. Valve plug 38 allows inflation fluid to flow in and out of central lumen 22 of hollow guidewire 20. When valve stem 30 is translated within hollow guidewire 20 relative to valve port 28 formed in a sidewall of hollow guidewire 20, plug valve 36 may be opened and closed, allowing fluid such as inflation fluid to be injected into and withdrawn from central lumen 22 to and from a distal end of hollow guidewire 20. When plug valve 36 is closed as shown, the flow of inflation fluid is blocked, for example, to prevent air or liquid from flowing through central lumen 22 or to keep an occlusion balloon inflated while in the body. When valve plug 38 is positioned across or distal to valve port 28, plug valve 36 is closed. When valve plug 38 is positioned proximal to valve port 28 as shown in FIG. 2b, plug valve 36 is open and fluid may flow through valve port 28 and central lumen 22 to and from a distal end of hollow guidewire 20.

FIG. 2b shows a longitudinal cross-sectional view of plug valve 36 of FIG. 2a in an open position, in accordance with one embodiment of the present invention. As valve stem 30 is translated to axially move valve plug 38 to a position proximal to valve port 28 and to open plug valve 36, inflation fluid 14 may be injected through valve port 28 and into central lumen 22 of hollow guidewire 20. Similarly, inflation fluid 14 may be withdrawn from central lumen 22 of hollow guidewire 20 through open valve port 28.

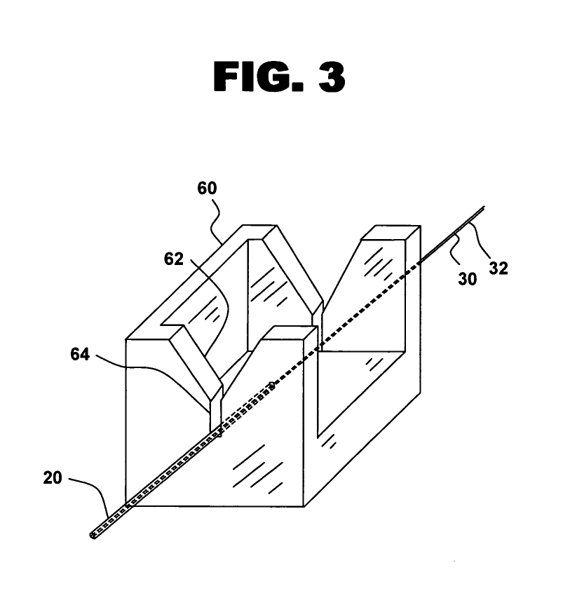

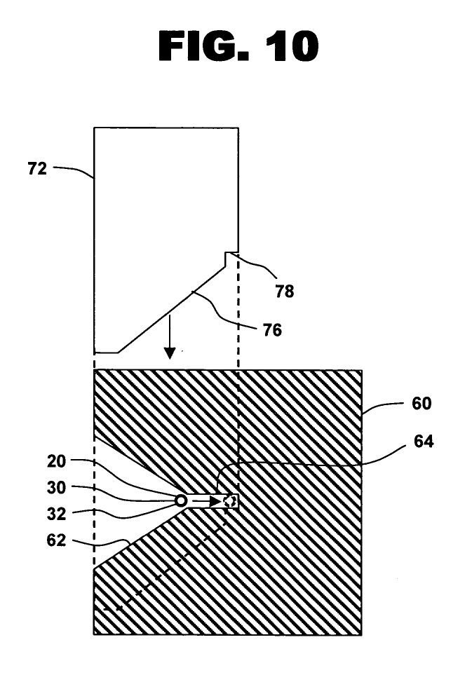

FIG. 3 shows a perspective view of medial v-groove alignment block 60, including v-groove 62 formed on one side thereof. V-groove 62 guides a transverse insertion of extended portion 32 of valve stem 30 into medial v-groove alignment block 60. Flanks of v-groove 62 slidably direct extended portion 32 of valve stem 30 into medial v-groove alignment block 60. Medial v-groove alignment block 60 may include slot 64 that extends from the apex of v-groove 62 to accommodate hollow guidewire 20 and valve stem 30. Slot 64, when used, is appropriately sized to allow the ready transverse insertion of extended valve stem 30 and a portion of hollow guidewire 20 into slot 64 to maintain hollow guidewire 20 in a position suitable for clamping. Medial v-groove alignment block 60 has a central recess to mate with tapered section 76 of jaw 72, illustrated in FIG. 10. The central recess eliminates material in medial v-groove alignment block 60 such that there is effectively a relatively short v-groove 62 at each end, with an optional slot 64 associated with each v-groove 62. One example of medial v-groove alignment block 60 has a height of 0.625 inches, a width of 0.252 inches, a length of 0.500 inches, a v-groove flank angle of 30 degrees, a slot width of 0.0189 inches, and a slot depth of 0.100 inches. The flanks of v-groove 62 and the v-grooves of other alignment blocks may be symmetrically configured about a center axis and having an included taper angle, for example, of 60 degrees. Alternatively, the flanks of v-groove 62 and other alignment blocks may be asymmetric about a center axis and having, for example, one flank at an angle and an opposing flank without any slanting or taper such as 45 degrees and 0 degrees, respectively.

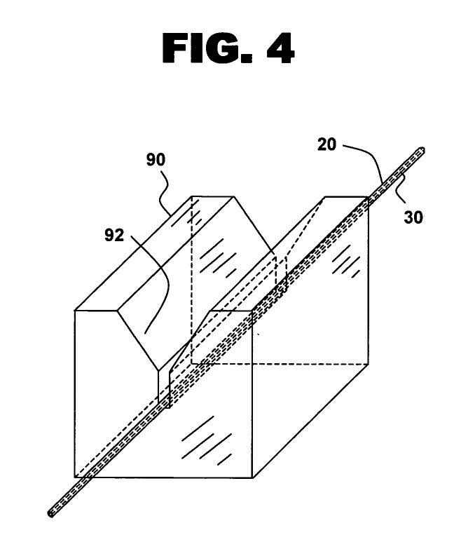

FIG. 4 shows a perspective view of a component of an inflation adaptor including a distal v-groove alignment block, in accordance with one embodiment of the present invention. Distal v-groove alignment block 90 includes v-groove 92 formed on one side of distal v-groove alignment block 90. V-groove 92 guides a transverse insertion of hollow guidewire 20 into distal v-groove alignment block 90. Flanks of v-groove 92 slidably direct a portion of hollow guidewire 20 into distal v-groove alignment block 90. Distal v-groove alignment block 90 may be slotted to accommodate hollow guidewire 20. The slot, when used, is appropriately sized to allow the ready transverse insertion of a portion of hollow guidewire 20 into the slot to maintain hollow guidewire 20 in a position suitable for clamping. One example of distal v-groove alignment block 90 has a length of nominally 0.400 inches with taper angles and slot dimensions that match the medial v-groove alignment block.

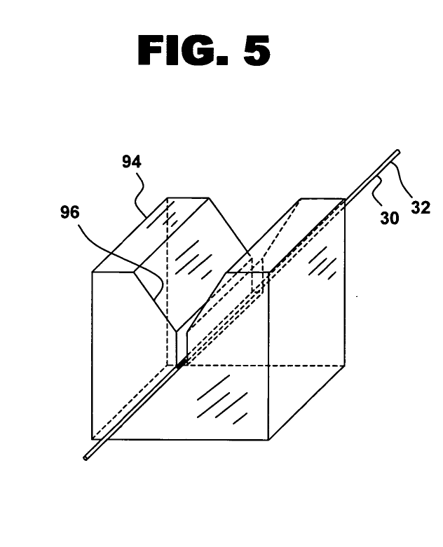

FIG. 5 shows a perspective view of a component of an inflation adaptor including a proximal v-groove alignment block, in accordance with one embodiment of the present invention. Proximal v-groove alignment block 94 includes v-groove 96 formed on one side of proximal v-groove alignment block 94. V-groove 96 guides a transverse insertion of extended portion 32 of valve stem 30 into proximal v-groove alignment block 94. Flanks of v-groove 96 slidably direct a portion of extended valve stem 30 into proximal v-groove alignment block 94. Proximal v-groove alignment block 94 may be slotted to accommodate extended portion 32 of valve stem 30. The slot, when used, is appropriately sized to allow the ready transverse insertion of a portion of valve stem 30 into the slot to maintain valve stem 30 in a position suitable for clamping. One example of proximal v-groove alignment block 94 has a length of nominally 0.300 inches with taper angles and slot dimensions that match the medial v-groove alignment block.

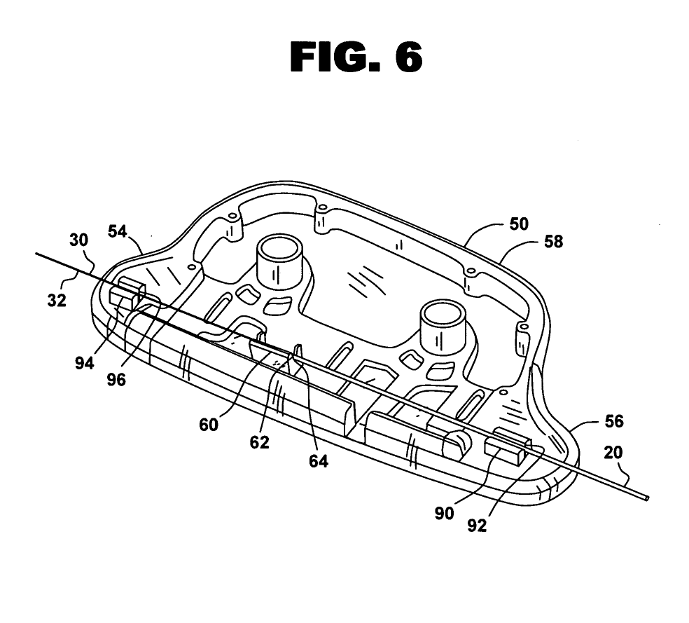

FIG. 6 shows an illustrative perspective view of an inflation adaptor including medial v-groove alignment block 60, distal v-groove alignment block 90, and proximal v-groove alignment block 94, in accordance with one embodiment of the present invention. Medial v-groove alignment block 60 is attached to or formed integrally with housing 58 of inflation adaptor 50. Alternatively, medial v-groove alignment block 60 may be attached to anvil 74. Medial v-groove alignment block 60 may include slot 64 that extends from the apex of v-groove 62 to accommodate hollow guidewire 20 and valve stem 30. Distal v-groove alignment block 90 with v-groove 92 is attached to or formed integrally with housing 58 near distal end 56 of inflation adaptor 50. Distal v-groove alignment block 90 is axially aligned with medial v-groove alignment block 60 to receive hollow guidewire 20. Proximal v-groove alignment block 94 with v-groove 96 is attached to or formed integrally with housing 58 near proximal end 54 of inflation adaptor 50. Proximal v-groove alignment block 94 is axially aligned with medial v-groove alignment block 60 to receive extended portion 32 of valve stem 30. Medial v-groove alignment block 60, distal v-groove alignment block 90, and proximal v-groove alignment block 94 are axially aligned and positioned within housing 58 of inflation adaptor 50 to allow hollow guidewire 20 and valve stem 30 to be positioned within inflation adaptor 50.

FIG. 7 shows a perspective view of jaw 72 for an inflation adaptor, in accordance with one embodiment of the present invention. Jaw 72 includes planar surface 68 with recesses for distal pad 80a and medial pad 82a. Jaw 72 includes an opening for sliding pad 84a. Jaw 72 is slotted on each side of tapered section 76 to accommodate a medial v-groove alignment block. Planar surface 68 may additionally include teeth, trapezoidal protrusions or other features for valve stem and hollow guidewire insertion.

FIG. 8 shows a perspective view of anvil 74 for an inflation adaptor, in accordance with one embodiment of the present invention. Anvil 74 is adapted with a cutout region to receive a medial v-groove alignment block, and contains recesses for distal pad 80b and medial pad 82b. Anvil 74 includes an opening for sliding pad 84b. Anvil 74 may include elongated longitudinally oriented channel 78 to accommodate a hollow guidewire with an extended valve stem.

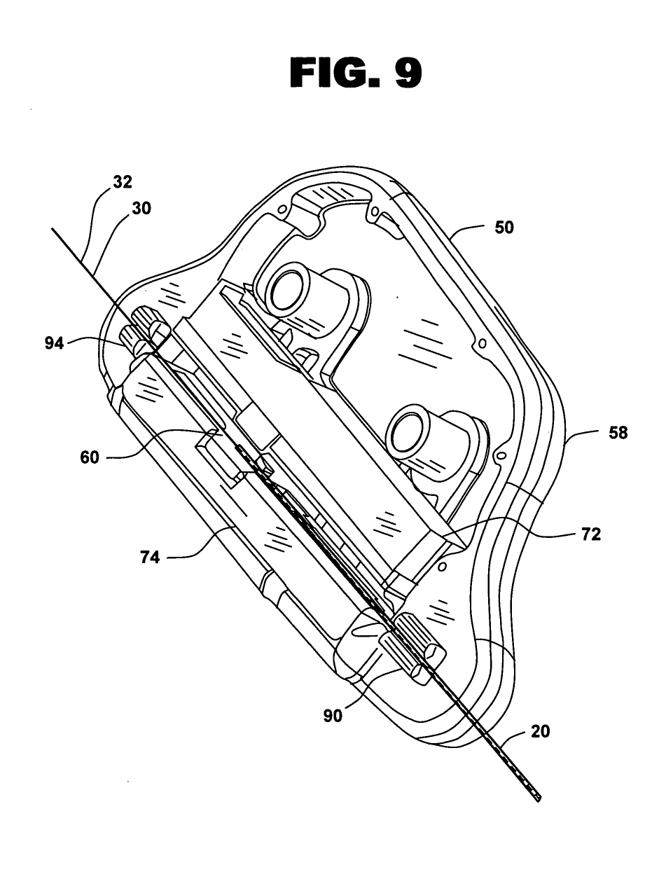

FIG. 9 shows an illustrative perspective view of inflation adaptor 50 including housing 58, medial v-groove alignment block 60, distal v-groove alignment block 90, proximal v-groove alignment block 94, jaw 72 and anvil 74, in accordance with one embodiment of the present invention. Inflation adaptor 50 includes medial v-groove alignment block 60 (not shown for sake of clarity) attached to housing 58 of inflation adaptor 50. Alternatively, medial v-groove alignment block 60 may be attached to anvil 74. Distal v-groove alignment block 90 is attached to or formed integrally with housing 58. Proximal v-groove alignment block 94 is attached to or formed integrally with housing 58. Jaw 72 and anvil 74 are positioned within inflation adaptor 50 to allow transverse insertion of hollow guidewire 20 with valve stem 30 into inflation adaptor 50. Extended portion 32 of valve stem 30 and hollow guidewire 20 are received in medial v-groove alignment block 60, distal v-groove alignment block 90 and proximal v-groove alignment block 94, and are engaged by a clamping device, including jaw 72 and anvil 74, for controlling flow of inflation fluid through a portion of hollow guidewire 20.

FIG. 10 shows a side view of medial v-groove alignment block 60 and tapered section 76 of jaw 72, in accordance with one embodiment of the present invention. Tapered section 76 of jaw 72 mates with medial v-groove alignment block 60. Tapered section 76 is moved into a recessed region of medial v-groove alignment block 60 to clamp hollow guidewire 20 with valve stem 30. As tapered section 76 of jaw 72 moves into medial v-groove alignment block 60, tapered section 76 slidably directs hollow guidewire 20 and valve stem 30 along flanks of v-groove 62 into slot 64. Channel 78 of tapered section 76 is positioned to contain extended portion 32 of valve stem 30 and hollow guidewire 20 when extended portion 32 of valve stem 30 and hollow guidewire 20 are positioned in medial v-groove alignment block 60.

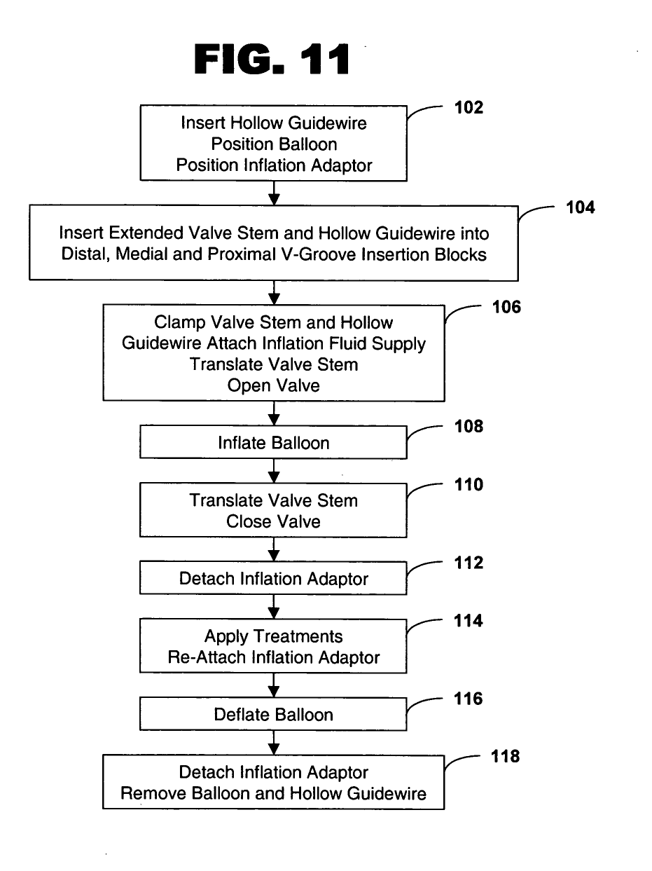

FIG. 11 shows a flow diagram of a method for operating an inflation adaptor, in accordance with one embodiment of the present invention. This method includes various steps to operate an inflation adaptor with a medial v-groove alignment block. The description pertains to the inflation and deflation of an inflatable balloon attached near the distal end of a balloon catheter, such as an occlusion catheter. Alternatively, the inflation adaptor operation method may be used to inflate and deflate an angioplasty balloon or to deploy a stent coupled to a stent-deployment balloon. Vascular condition treatment methods employing the inflation adaptor provide, for example, one or more vascular treatments for the prevention or correction of various ailments and deficiencies including those associated with the cardiovascular system, the cerebrovascular system, and other blood vessels within the body.

A balloon catheter with a hollow guidewire and an inflatable balloon is inserted and positioned in a bodily vessel, as seen at block 102. The hollow guidewire with the inflatable balloon near its distal end is manipulated manually through the vascular system to the desired location for placement of the balloon. For example, a needle puncture is made in the body near the femoral artery, and the hollow guidewire with the inflatable balloon is inserted through the puncture, through the femoral artery, and into a position within a blood vessel where the balloon may be inflated to block fluid flow in the vessel. A central lumen within the hollow guidewire and other lumens may be purged with inflation fluid such as diluted contrast fluid or saline solution before the balloon catheter is inserted into the body. Prior to the positioning of the balloon catheter, fluoroscopic contrast fluid may be injected into the blood vessel in order to identify, visualize and verify the location of a stenosis, blockage, or other medical condition within the blood vessel. In one example, the hollow guidewire and the inflatable balloon are advanced through a vessel and positioned distal to the site of a stenosis.

When the balloon and the hollow guidewire have been appropriately positioned, the inflation adaptor is positioned to receive a valve stem partially extended from a proximal end of the hollow guidewire to define a first valve configuration, such as a closed position. A portion of the valve stem with an attached valve plug extends into a proximal portion of the hollow guidewire, forming a plug valve that controls the flow of fluid through a valve port in a sidewall of the hollow guidewire and into the inflatable balloon near the distal end of the hollow guidewire.

To attach the inflation adaptor to the balloon catheter, the extended valve stem is transversely inserted and positioned into one or more alignment blocks having v-grooves, as seen at block 104. One embodiment of the present invention has a medial v-groove alignment block, a distal v-groove alignment block and a proximal v-groove alignment block. The extended portion of the valve stem and the proximal end of the hollow guidewire are transversely inserted into a v-groove of the medial v-groove alignment block as the hollow guidewire is transversely inserted into a v-groove of the distal v-groove alignment block and the extended portion of the valve stem is transversely inserted into a v-groove of the proximal v-groove alignment block.

In another embodiment of the present invention, which has neither the distal v-groove alignment block nor the proximal v-groove alignment block, the extended valve stem and the hollow guidewire are transversely inserted and positioned into the v-groove of the medial v-groove alignment block and into the medial v-groove alignment block. Another embodiment of the present invention employs a distal v-groove alignment block in conjunction with the medial v-groove alignment block. In yet another embodiment, a proximal v-groove alignment block is used in conjunction with the medial v-groove alignment block. In all aforementioned embodiments of the present invention, the valve port in the hollow guidewire is appropriately positioned and aligned with an inflation fluid supply port within the inflation adaptor using, for example, metallic or colored markers, indicia, or other suitable indicators for the valve port.

A portion of the extended valve stem and the hollow guidewire are clamped, as seen at block 106. The valve stem and the hollow guidewire are axially translated with respect to each other to a second valve configuration to control flow of inflation fluid through a portion of the hollow guidewire. For example, a clamping device within the inflation adaptor is actuated by rotating an actuation knob from a first position that allows the transverse insertion of the extended valve stem and hollow guidewire to a second position that activates a clamping device to engage the extended valve stem and the hollow guidewire. Clamping the extended portion of the valve stem and the hollow guidewire comprises, for example, engaging the hollow guidewire with a set of distal pads and a set of proximal pads, and engaging the extended valve stem with a set of sliding pads. When the valve stem and the hollow guidewire are clamped, seals around the valve port in the hollow guidewire are secured, allowing pressurized fluid from an inflation fluid supply to be injected into the valve port and through the hollow guidewire.

At this point in the method, an inflation fluid supply is coupled to the inflation adaptor. Inflation fluid such as dilute contrast agent or other suitable fluid may be contained in an inflation device and connected to an inflation fluid port on the inflation adaptor, such as a Luer fitting. Various standard procedures can be used to remove air and other gases from the inflation device, inflation fluid lines connected to the inflation adaptor, and chambers within the inflation adaptor. Alternatively, the inflation fluid supply may be connected to the inflation adaptor prior to attaching the inflation adaptor to the balloon catheter, prior to inserting the hollow guidewire into the body, or at other times during treatment, depending on the preference of the medical practitioner.

The valve stem is translated relative to the hollow guide wire when in the clamped position. Flow of an inflation fluid through a portion of the hollow guidewire is controlled based on the relative translation. For example, a plug valve, which includes a polymeric valve plug attached near a distal end of a portion of the valve stem that extends into the hollow guidewire, is operated by axially displacing the valve stem with respect to the hollow guidewire. Axial translations of the valve stem relative to the hollow guidewire block allow the flow of inflation fluid through a valve port in the side of the hollow guidewire. When in a closed position, the valve plug is located distal to the valve port and prevents the flow of fluid through a guidewire central lumen. When in an open position, the valve plug is located proximal to the valve port, allowing fluid to flow through a portion of the hollow guidewire and into or out from an inflatable balloon near the distal end of the hollow guidewire. The plug valve may be opened, for example, by rotating an actuation knob of the inflation actuator from the second position to a third position, which translates the valve stem with respect to the valve stem to an open valve position.

The inflatable balloon proximate to a distal end of the hollow guidewire is inflated, as seen at block 108. The inflatable balloon is inflated by forcing inflation fluid from the inflation fluid supply through a fluid supply port in the inflatable adaptor, through the valve port in the sidewall of the hollow guidewire, through the guidewire central lumen, through one or more balloon inflation holes of the inflatable balloon, and into an interior region of the inflatable balloon. The inflatable balloon is inflated with the injected inflation fluid to the desired size, which may be monitored with injections of radiopaque contrast fluid and/or with associated x-ray imaging systems. Following the inflation of the balloon, an angiogram using fluoroscopy may be taken to ensure the complete occlusion of a vessel by the balloon.

When the inflatable balloon is inflated to the desired diameter and is occlusively apposed or anchored to the vessel wall, the valve stem within the hollow guidewire is axially translated to close the plug valve, as seen at block 110. Translation of the valve stem within the hollow guidewire is achieved, for example, by rotating the actuation knob from the third position back to the second position.

Inflation fluid within the inflatable balloon is retained to keep the inflatable balloon inflated and the inflation adaptor is detached, as seen at block 112. The inflation adaptor is detached by unclamping the valve stem and hollow guidewire, and then sliding and removing the inflation adaptor from the valve stem and hollow guidewire. The valve stem and hollow guidewire are unclamped, for example, by rotating the actuation knob from the second position back to the first position.

When the inflation adaptor is detached, one or more treatments may be applied to the vessel, as seen at block 114. In one treatment example, a balloon dilation catheter may be advanced over the hollow guidewire to the treatment site where angioplasty is performed. After the restriction has been treated, the primary treatment catheter may be removed from over the hollow guidewire and then an aspiration catheter can be advanced to the treatment site to aspirate any embolic debris generated during the angioplasty. Other types of catheters such as imaging catheters, over-the-wire treatment catheters, rapid exchange catheters, stent deployment catheters, inspection catheters or other types of catheters may be used in conjunction with the hollow guidewire.

When one or more treatments have been completed, the inflation adaptor is reattached. The hollow guidewire with the extended valve stem is transversely inserted through the receiver(s) of the one or more alignment blocks within the inflation adaptor. After being properly aligned within the inflation adaptor, the hollow guidewire and the extended valve stem are clamped. Once clamped, the extended valve stem is again translated with respect to the hollow guidewire to slide the valve plug past the valve port and open the valve.

The inflatable balloon is deflated, as seen at block 116. The balloon may be deflated when inflation fluid within the inflatable balloon is drawn out or when it is forced out, for example, by elastic restoring forces exerted on the inflation fluid within the interior region by the balloon material. The inflatable balloon may be deflated, for example, with an inflation/deflation device coupled to the inflation adaptor.

The inflation adaptor may be detached prior to removal of the hollow guidewire, as seen at block 118. Once the inflatable balloon is deflated, the plug valve is axially translated into a closed position. The inflation fluid supply may be disconnected from the inflation fluid supply port on the inflation adaptor. The hollow guidewire and the valve stem are unclamped, and the inflation adaptor is readily removed. The balloon catheter with the hollow guidewire and the inflatable balloon may be repositioned within the body or removed from the body and discarded. When the inflation adapter is used to inflate and deflate the balloon of an angioplasty or stent delivery catheter, steps 110 through 114 are omitted.

In addition to the inflation adaptor being used in treatments employing occlusion balloon catheters, angioplasty catheters, balloon dilatation catheters and stent-deployment catheters, it also may be used in other applications such as the deployment of emboli filters and other procedures utilizing controlled axial translations of a wire within a small-diameter hollow tube.

Variations and alterations in the design, manufacture and methods of use of the inflation adaptor are apparent to one skilled in the art, and may be made without departing from the spirit and scope of the present invention. While the embodiments of the invention disclosed herein are presently considered to be preferred, various changes and modifications can be made without departing from the spirit and scope of the invention. The scope of the invention is indicated in the appended claims, and all changes that come within the meaning and range of equivalents are intended to be embraced therein.

1. A system for treating a vascular condition, comprising:
an inflation adaptor including a housing;
a clamping device positioned within the housing, the clamping device including a jaw and an anvil; and
a medial v-groove alignment block; wherein a portion of an extended valve stem and a hollow guidewire are received in the medial v-groove alignment block and are engaged by the clamping device to allow the extended valve stem and the hollow guidewire to be axially translated relative to each other to control a flow of an inflation fluid through the hollow guidewire when the clamping device is in a clamped position.

2. The system of claim 1 wherein the jaw has a planar surface.

3. The system of claim 1 wherein the jaw includes a tapered section having a shoulder to contain the extended valve stem and the hollow guidewire when the extended valve stem and the hollow guidewire are positioned in the medial v-groove alignment block.

4. The system of claim 1 wherein the medial v-groove alignment block is attached to the anvil.

5. The system of claim 1 wherein the anvil is adapted to receive the medial v-groove alignment block.

6. The system of claim 1 wherein the medial v-groove alignment block is attached to or formed integrally with the housing.

7. The system of claim 1 wherein the anvil includes an elongated longitudinally oriented channel.

8. The system of claim 1 wherein the clamping device includes a set of distal pads and a set of medial pads to engage the hollow guidewire received in the medial v-groove alignment block.

9. The system of claim 1 wherein the clamping device includes a set of sliding pads to engage the extended valve stem and to control the axial translation of the valve stem relative to the hollow guidewire.

10. The system of claim 1 further comprising:
a distal v-groove alignment block attached proximate to a distal end of the inflation adaptor, wherein the distal v-groove alignment block is axially aligned with the medial v-groove alignment block to receive the hollow guidewire.

11. The system of claim 1 further comprising:
a proximal v-groove alignment block attached proximate to a proximal end of the inflation adaptor, wherein the proximal v-groove alignment block is axially aligned with the medial v-groove alignment block to receive the extended valve stem.

12. The system of claim 1 further comprising:
a multi-position actuation knob coupled to the clamping device, wherein a first position of the actuation knob allows insertion of the extended valve stem and the hollow guidewire into the clamping device, and wherein moving the actuation knob from the first position to a second position of the actuation knob activates the clamping device to engage the extended valve stem and the hollow guidewire, and wherein moving the actuation knob from the second position to a third position of the actuation knob translates the valve stem relative to the hollow guidewire to control the flow of the inflation fluid into the inflatable balloon.

13. The system of claim 1 further comprising:
an inflation fluid supply port, wherein the inflation fluid from an inflation fluid supply connected to the inflation fluid supply port is injected through a portion of the hollow guidewire when the clamping device is in a clamped position and a plug valve within the hollow guidewire is in an open position.

14. The system of claim 1 further comprising:
an inflatable balloon attached proximate to a distal end of the hollow guidewire; wherein a portion of the extended valve stem and the hollow guidewire are received in the medial v-groove alignment block and are engaged by the clamping device to allow the valve stem and the hollow guidewire to be axially translated relative to each other to control the flow of the inflation fluid into the inflatable balloon.

15. The system of claim 14 wherein the inflatable balloon comprises one of an occlusion balloon, an angioplasty balloon, and a stent-deployment balloon.

16. The system of claim 14 further comprising:
a plug valve having a valve plug attached to a portion of the valve stem positioned within a central lumen of the hollow guidewire, wherein the plug valve is positioned in one of an open position or a closed position when the valve stem and the hollow guidewire are translated relative to each other to control the flow of the inflation fluid into the inflatable balloon.

17. The system of claim 14 further comprising:
a multi-position actuation knob coupled to the clamping device, wherein a first position of the actuation knob allows insertion of the extended valve stem and the hollow guidewire into the clamping device, and wherein moving the actuation knob from the first position to a second position of the actuation knob activates the clamping device to engage the extended valve stem and the hollow guidewire, and wherein moving the actuation knob from the second position to a third position of the actuation knob translates the valve stem relative to the hollow guidewire to control the flow of the inflation fluid into the inflatable balloon.

18. The system of claim 14 further comprising:
an inflation fluid supply coupled to the inflation adaptor, wherein the inflation fluid from the inflation fluid supply is injected through a portion of the hollow guidewire into an interior region of the inflatable balloon when the clamping device is in a clamped position and a plug valve within the hollow guidewire is in an open position.

19. A method of operating an inflation adaptor, comprising:
positioning the inflation adaptor to receive a valve stem partially extended from a proximal end of a hollow guidewire to define a first valve configuration;
inserting the extended valve stem and the hollow guidewire into a medial v-groove alignment block;
clamping respective portions of the extended valve stem and the hollow guidewire within the inflation adaptor; and
relatively translating the valve stem and the hollow guidewire to a second valve configuration to control a flow of an inflation fluid through a portion of the hollow guidewire.

20. The method of claim 19 wherein inserting the extended valve stem and the hollow guidewire into the medial v-groove alignment block comprises slidably directing the extended valve stem and the hollow guidewire into the medial v-groove alignment block.

21. The method of claim 19 wherein clamping the respective portions of the extended valve stem and the hollow guidewire comprises engaging the valve stem with a set of distal pads and a set of medial pads, and engaging the hollow guidewire with a set of sliding pads.

22. The method of claim 19 wherein the first valve configuration is a closed valve position.

23. The method of claim 19 wherein the second valve configuration is an open valve position.

24. The method of claim 19 further comprising:
inserting the hollow guidewire into a distal v-groove alignment block.

25. The method of claim 19 further comprising:
inserting the extended valve stem into a proximal v-groove alignment block.

26. The method of claim 19 further comprising:
coupling an inflation fluid supply to the inflation adaptor.

27. The method of claim 19 further comprising:
inflating an inflatable balloon attached proximate to a distal end of the hollow guidewire while in the clamped position.

28. The method of claim 19 further comprising:
deflating an inflatable balloon attached proximate to a distal end of the hollow guidewire while in the clamped position.
